(19) United States
(12) Patent Application Publication
(10) Pub. No.: US 2007/0232889 A1
(43) Pub. Date: Oct. 4, 2007

(54) Method for imaging an infarction patient's myocardium and method for supporting a therapeutic intervention on the heart

(75) Inventors: Jan Boese, Eckental (DE); Norbert Rahn, Forchheim (DE); Stefan Lautenschlager, Hausen (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Aktiengesellschaft

(21) Appl. No.: 11/729,657

(22) Filed: Mar. 29, 2007

(30) Foreign Application Priority Data
Mar. 30, 2006 (DE) 10 2006 014 882.7

(51) Int. Cl. A61B 5/05

(52) U.S. Cl. 600/407

(57) ABSTRACT

Two 3D image data records are obtained mutually independently comprising healthy myocardium, myocardium having a reduced blood supply, and the necrotic myocardium. The image data records are combined to produce an overall image data record after registration, and 2D image representations are produced from the overall image data record in which the necrotic parts of the myocardium are shown emphasized, with simultaneously showing the endocardium or the healthy parts of the myocardium and parts having a reduced blood supply. The overall image data record can be used afterwards. For example, further registering step is carried out using images obtained during an intervention on the myocardium. The further registering step enables the necrotic parts of the myocardium to be assigned to the patient's situation. That can extend as far as catheters being moved automatically up to a boundary of the necrotic myocardium.

### CROSS REFERENCE TO RELATED APPLICATIONS

This application claims priority of German application No. 10 2006 014 882.7 filed Mar. 30, 2006, which is incorporated by reference herein in its entirety.

### FIELD OF THE INVENTION

The invention relates to a method for imaging an infarction patient's myocardium. It relates also to a method for supporting a therapeutic intervention on the heart.

### BACKGROUND OF THE INVENTION

The myocardium (heart tissue) can be damaged during a myocardial infarction. A distinction is made following a myocardial infarction between healthy myocardium being supplied normally with blood, myocardium that has a reduced blood supply (but is not yet necrotic), and necrotic myocardium that is dead.

Cardiological interventions or, later, also electrophysiological procedures are frequently carried out following an acute myocardial infarction in order to treat the patient. It is not possible to treat necrotic myocardial tissue. The starting point is as a rule the myocardial tissue having a reduced blood supply. Areas of myocardial tissue having a reduced blood supply can be treated by expanding narrowed coronary arteries. Although it is known how to prepare for said kind of interventions by imaging the coronary arteries—for imaging, a contrast medium is precision-injected into the coronary arteries by means of a catheter inserted into the heart—it is not possible, using said imaging results, to answer the question as to which part of a coronary artery is to be selectively treated in order to improve the blood supply to myocardial tissue having a reduced blood supply. No information can be gained during an intervention about whether further coronary arteries or further sub-branches of the artery are to be treated. As the cited image shows only the coronary artery itself, nor can the outcome of the intervention be inspected to check whether tissue previously having a reduced blood supply is actually being better supplied with blood.

Electrophysiological ablating (obliterating of tissue) can also be applied for treating cardiac rhythm disturbances. It is of practical advantage during ablating for the necrotic regions to be known, because ablating will be especially effective when tissue that is in the vicinity of necrotic tissue and causing impairments of conduction is obliterated.

The doctors providing treatment have hitherto had to rely on empirical values. There is no supporting imaging method that provides a representation in which necrotic tissue and tissue having a reduced blood supply are imaged simultaneously adequately well.

Necrotic myocardial tissue can be visualized on its own, specifically by means of nuclear magnetic resonance imaging or computed tomography (see Andreas H. Mahnken et al., “Assessment of myocardial viability in reperfused acute myocardial infarction using 16-slice computed tomography in comparison to magnetic resonance imaging”, Journal of the American College of Cardiology, Vol. 45, No. 12, 21. June 2005, pp. 2042 to 2047).

SPECT (single photon emission computer tomography) can also be employed, see J-F. Paul, M. Wartski, C. Caussin, et al.: Late Defect on Delayed Contrast-enhanced Multi-Detector Row CT Scans in the Prediction of SPECT Infarct Size after Reperfused Acute Myocardial Infarction: Initial Experience”, Radiology, 236, pp. 485 to 489, Jun. 21, 2005. PET (positron emission tomography) can also be used for imaging necrotic myocardial tissue (see C. Klein, et al., “Assessment of myocardial viability with contrast enhanced magnetic resonance imaging: comparison with positron emission tomography”, Circulation, 105(2), pp. 162 to 167, Jan. 15, 2002).

However, with none of said imaging methods is tissue having a reduced blood supply visualized adequately well alongside the necrotic myocardial tissue.

### SUMMARY OF THE INVENTION

The object of the invention is to support a doctor providing treatment by providing suitable imaging, for example of the entire myocardium.

Said object is achieved by a method as claimed in the claims. Said method is developed by two further methods for supporting a therapeutic intervention on the heart as claimed in the claims.

The inventive method begins at step a1): Producing a first 3D image data record (of the heart) immediately after administration of a contrast medium to the patient. What is to be understood by “immediately after administration” is that the customary action time necessary for obtaining image information about the endocardium and about healthy parts of the myocardium and paths thereof having a reduced blood supply is maintained. It is therein basically possible to employ any imaging method using the appropriate contrast medium. Computer tomography angiography, nuclear magnetic resonance angiography, or X-ray rotation imaging will typically be employed.

The method is continued at step a2): Segmenting the image data record. The segmenting of an image data record is as such the prior art. The intended result of a segmenting step is for the image data record to be divided into two image data records by distinguishing between different objects imaged therein. Segmenting is typically done by using threshold criteria in conjunction with what is termed “region growing”, wherein the threshold criterion is applied point-by-point proceeding from a starting point, with a growing region being assigned respectively checked points or not.

The result of segmenting the image data is in the present case obtaining at least one of two separate image data records, namely:
an endocardium image data record containing image information about the endocardium, and/or
a myocardium image data record containing image information about the myocardium.

The endocardium is the boundary area between the myocardium and the blood in the cardiac ventricle. Segmenting is made easier through there being only blood in the endocardium (together with the contrast medium), while although the myocardium contains blood, this does not completely fill the space because that is, of course, occupied by the tissue being supplied with blood in partial volumes only.

The inventive method is continued as follows: A second 3D image data record is produced when a pre-specified period of time has elapsed after a contrast medium has been administered to the patient, with image information being therein obtained about necrotic parts of the myocardium. That is because it is a common feature of the above-cited methods for obtaining image information about necrotic parts of the myocardium that a contrast medium must already have left the main parts of the heart so that blood mass and healthy parts of the myocardium will not be imaged. The contrast medium usually collects in the necrotic parts of the myocardium so that when a pre-specified period of time has elapsed, usually ten minutes (5 to 15 minutes), only the necrotic parts of the myocardium will be imaged.

Information on the one hand about the endocardium or the healthy myocardium and that having a reduced blood supply and, on the other, about the necrotic parts of the myocardium has hitherto been available separately.

This is combined as follows:

What is termed registering is carried out at step c). The term registering, which is known as such in the prior art, entails assigning one image data record's 3D image data to another image data record's 3D image data accurately in terms of position and dimensions. That is necessary because, having been produced mutually independently, the different 3D image data records have to a certain extent different “systems of coordinates”. Registering means nothing other than that an image of one system of coordinates is superimposed onto the other such system. Registering, which is the subject of numerous publications relating to the prior art, includes image recognition: So that the individual 3D image data records can be assigned to each other, it is necessary for common structures imaged therein to be recognized. That is enabled chiefly by the fact that although mainly the necrotic parts of the myocardium are imaged in the image produced at step b), there is still weak residual imaging of the healthy myocardium and of that having a reduced blood supply and, possibly, of the endocardium. There are hence no major obstacles to registering.

The mutually assigned (registered) image data records are then at step d) mutually superimposed to produce an overall 3D image data record.

The concluding step of the inventive method is step e): Overall 2D image data representations (for the purpose of, for example, 3D image visualizing) are produced in which are imaged on the one hand the endocardium or the healthy parts of the myocardium and parts thereof having a reduced blood supply and, on the other, the necrotic parts of the myocardium. The two-dimensional representations of 3D data records can basically be 2D layer images or else exterior 2D views in the case of which a 3D space can be traversed by, for instance, moving a mouse.

The inventive method thus achieves the aim of providing an image in which the necrotic parts of the myocardium are discernible, with other structures (endocardium or, as the case may be, healthy parts of the myocardium and parts thereof having a reduced blood supply) being imaged simultaneously, with the aid of which the doctor providing treatment can form a spatial picture. Only through seeing the joint image will the doctor providing treatment know where the necrotic parts of the myocardium are precisely located in the heart.

The representation is preferably such that the doctor will be able to distinguish between healthy myocardium, myocardium having a reduced blood supply, and necrotic myocardium.

By virtue of a threshold criterion it is for that purpose possible at step a₂) to distinguish between healthy parts in the myocardium image data record and parts therein having a reduced blood supply. The different parts are then each assigned different attributes. An attribute can be a simple numerical value between 0 and 1. Simplifying, the use of binary values is even possible. The necrotic parts of the myocardium are at step b) likewise each assigned an attribute differing from that of the other parts of the myocardium. For example a numerical value of between 0 and 0.33 can signify healthy myocardium, a numerical value of between 0.33 and 0.67 can signify myocardium having a reduced blood supply, and a numerical value of between 0.67 and 1 can signify a necrotic myocardium. Image data having different attributes is then at step e) shown in different ways. For example a gray-scale value can be proportional to the numerical value in the attribute. Differentiation can, though, be such that there is a clear delineation between parts of the myocardium having a reduced blood supply and necrotic parts thereof by, for example, showing healthy myocardium, myocardium having a reduced blood supply, and necrotic myocardium each in different colors. It will thus be made easier for the doctor providing treatment to recognize the necrotic myocardium in the representation compared to the parts of the myocardium having a reduced blood supply.

Registering step c) is in a preferred embodiment made easier to perform by, when the first and second 3D image data record are being produced, allowing for the cardiac phase and, preferably simultaneously, the respiration phase. That is routinely done by producing an electrocardiogram (ECG), with the signals obtained with the aid of said ECG being temporally assigned to the 3D image data produced, and with respectively only the 3D image data associated with a pre-specified cardiac and/or respiration phase being included at recording steps a₁,) and b) in the 3D image data record. If the same pre-specified cardiac or, as the case may be, respiration phase is respectively used in steps a₁ ) and b), then the image structures will be especially clear in the image data and registering step c) will consequently be particularly uncomplicated to perform.

The previous method related to producing a pre-operative overall image data record. The specific feature therein constituting an advance is that the doctor will be supported through visualizing during a therapeutic intervention. It is customary for images to be produced as an augment during therapeutic interventions on the heart. In a preferred development, a further method is provided for supporting a therapeutic intervention on the heart as claimed in the claims. With said method, first (k) steps a1) up to d) is carried out, which is to say the pre-operative overall image data record is obtained, specifically by means of images before the therapeutic intervention.

Image information is then obtained at step l) during the therapeutic intervention. The image information obtained at step l) can be multifarious. Individual two-dimensional X-ray images can be obtained, or a few, mutually associated two-dimensional X-ray images that are combined into one, three-dimensional overall image. Finally, the novel method of 3D cardiac rotation angiography described in DE 10 2005 016472.2 (published subsequently) can be applied.

Step l) is not, though, restricted to the production of X-ray images. Rather it is also possible to obtain three-dimensional electroanatomical mapping data and visualize this in the form of two-dimensional images (maps). Reference is made in this connection to, for example, the Carto mapping system from the company Biosense Webster. An electroanatomical map is derived from measurements which a catheter having a signal-recording unit obtains at different sites, with the signal strength or a signal-time curve being assigned to the respective site. Information of value to the doctor can be gained through imaging of the signal strengths or signal-time curves. The obtaining of electroanatomical maps is also described in, for example, DE 103 40 544 A1.

The further method for supporting a therapeutic intervention on the heart is continued at step m): Registering is repeated. This time the operation image data obtained at step l) is registered with the entire image data record. Registering is to be understood here, too, as meaning that the 2D or 3D image data of the operation images or, as the case may be, of the operation image data record is assigned to the 3D image data of the overall image data record accurately in terms of position and dimensions. Since common image structures have to be detected during registering, it is expedient at step k) to carry out the method in such a way that the endocardium image data record is used because the endocardium can be seen particularly well in X-ray images produced during the therapeutic intervention. The registering of 2D with 3D image data is as such perfectly possible, see for example J. Weese, T. M. Buzug, G. P. Penney, P. Desmedt, “2D/3D Registration and Motion Tracking for Surgical Interventions”, Philips Journal of Research 51 (1998), pp. 299 to 316.

Operation images are then at step n) superimposed on at least a part of the image data of the overall image data record. As a part of the image data of the overall image data record it is expedient to select the necrotic myocardium, which should preferably in parts be distinguished through attribute assigning from the other image data. It can moreover also be discernible in the image data of the overall image data record which image data goes back to the second 3D image data record (step b) above).

At concluding step o), 2D image representations are then produced in which at least the pre-operatively recorded necrotic myocardium is shown superimposed on an operation image representation going back to step l). In other words the pre-operatively recorded myocardium is shown superimposed on an image of the situation during the therapeutic intervention. A representation of such kind is also totally novel. Compared with the previously method having the image-producing step e), the further method constitutes yet a further improvement because the correlation between the necrotic myocardium and the actual situation during the operation has here been established for the doctor.

The detailed image data further permits the following additional invention: The coordinates of a boundary between the necrotic myocardium and other areas of tissue are determined using the image representations produced at step o) (or, if o) is omitted, using the superimposed image data obtained at step n)). Registering having, of course, taken place at step m), that is done in the patient system used during the intervention, even if the necrotic myocardium has been pre-operatively recorded. That will make it possible for a therapy instrument (for example an ablation catheter) to be moved to at least one point on the boundary under automatic control (by means of a suitable control motor). The ablation catheter can preferably even be moved along the entire boundary. As mentioned in the introduction, the boundary between the necrotic myocardium and the parts thereof having a reduced blood supply is of interest particularly for ablating because, on the one hand, ablating aimed at restoring the blood supply to the necrotic myocardium will serve little purpose as that is by definition already dead; on the other hand, ablating specifically at the boundary between necrotic myocardium and myocardium having a reduced blood supply will be to particularly good purpose. The inventive method can thus be advanced to the extent that the doctor can let the system operate autonomously and will only have to intervene supportively.

For implementing automatic guiding of a therapy instrument it is not necessary for electroanatomical maps to have been obtained directly at step l). Rather it is the case that conventional X-ray images can be obtained at step l)—with, owing to a fixed coordinate relationship with the catheter system, no further registering being required—in order then directly to assign electroanatomical maps to the necrotic myocardium after registering step m).

In other words it is possible at step n) alternatively also to superimpose other images, provided these have a fixed spatial relationship with the operation images, on a part of the image data of the overall image data record.

The therapeutic intervention cited in the further method does not of necessity have to be ablating. The method can be used also for supporting cardiological interventions such as, for example, stenting constricted coronary arteries. Images in which the coronary arteries are shown must for that purpose be obtained at step l). That is done preferably by at step l) producing 2D angiograms or 3D rotation angiograms using a C-arm X-ray system once a contrast medium has been injected into the coronary arteries with the aid of a catheter, with steps m) to o) thereafter requiring to be carried out.

Whereas registering step c) is necessary since registering has to be especially precise so that the different areas of the myocardium can be differentiated in the final presentation, registering step m) is not absolutely essential in the further method. The invention thus alternatively provides another further method. The overall image data record (steps a1) to d) is produced here, too, according to step k), with the first and second 3D image data record now being produced using a C-arm X-ray system. Registering is rendered superfluous through the patient's then being left, for performing step l), in a fixed position in the C-arm X-ray system, and through the same C-arm X-ray system's being used during the therapeutic intervention. Step l) in the another further method is accordingly as follows: With a contrast medium having been injected into the coronary arteries by means of a catheter, using the same C-arm X-ray system to record data for one or more 2D angiograms or 3D rotation angiograms to obtain image information about the coronary arteries. Steps n) and o) follow directly on from step l) without the need for a registering step of the nature of step m). Registering would at most be necessary if the patient had moved a considerable extent.

The result here, too, is that 2D images are produced in which the healthy parts of the myocardium, those having a reduced blood supply, and necrotic parts thereof are shown as well as, simultaneously, the coronary arteries. A representation of said kind will enable the doctor to treat the specific coronary arteries supplying blood to myocardium having a reduced blood supply in order to revive said myocardium.

The same C-arm X-ray system being used, it is almost obvious to repeat steps k) and l) for the purpose of inspecting the outcome, for example. The doctor providing treatment will then be able tell directly from the image whether an area of the myocardium having a reduced blood supply is now being better supplied with blood as a result of the treatment performed on the coronary artery.

In the further method or, as the case may be, the another further method it is preferably provided here, too, for the image data to be selected using the ECG (ECG gating). Thus only image data belonging to the same pre-specified cardiac and/or respiration phase should respectively be used at step a₁,) and b) (of step k)) and at step l). A particularly clear image representation will be achieved thereby. Registering step m) is simplified in the further method. The images are easier to superimpose in the another further method.

### BRIEF DESCRIPTION OF THE DRAWINGS

Preferred embodiments of the inventions are described below with reference to the drawing, in which:

FIG. 1 shows an image resulting from the inventive method,

FIG. 2 presents an alternative to the image representation shown in FIG. 1,

FIG. 3 presents a development of the image representation shown in FIG. 2, and

FIG. 4 presents a development of the image representation shown in FIG. 1.

### DETAILED DESCRIPTION OF THE INVENTION

In the inventive method a 3D image data record is first produced in which can be seen endocardium, healthy parts of the myocardium, and parts thereof having a reduced blood supply.

A contrast medium is therein used in the conventional manner. An image representation of said kind is known from the prior art.

It is further known from the prior art cited in the introduction how to produce a second 3D image data record in which the necrotic myocardial regions are particularly well imaged. Although a contrast medium is employed for that purpose, a period of time is allowed to elapse until said medium has dispersed. As the contrast medium collects mainly in the necrotic parts of the myocardium, those are particularly well imaged.

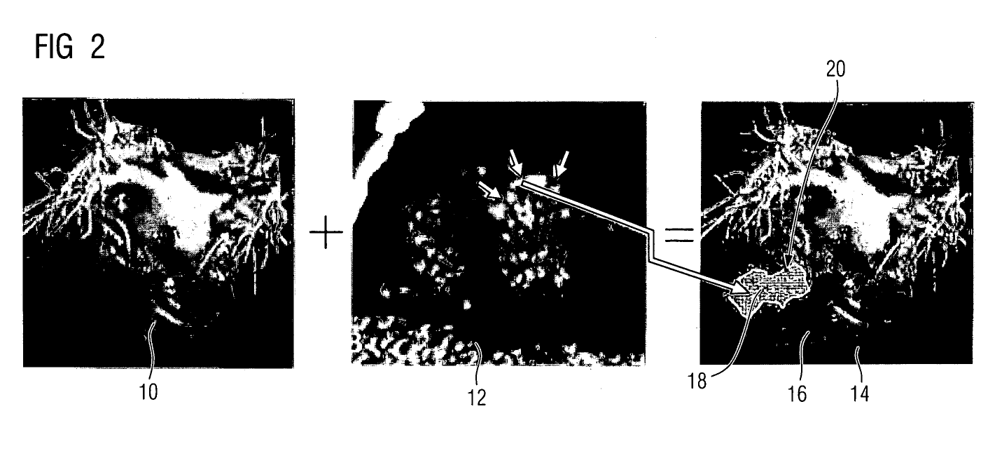

Two 3D image data records are hence available. These are then to be used for producing a superimposed representation. The first 3D image data record produced is for that purpose segmented. The method of segmenting is as such known in the prior art. What is obtained through segmenting is an endocardium image data record containing image information about the endocardium. A two-dimensional representation of said endocardium image data record can be found, for example, as image 10 in FIG. 2. Obtained additionally or alternatively is a myocardium image data record containing image information about the myocardium (namely the healthy tissue and that having a reduced blood supply). A difference between the different types of myocardium in the myocardium image data record can at that time be determined simultaneously using threshold criteria, so that healthy tissue and that having a reduced blood supply can then be differentiated. For the purpose of distinguishing, an attribute is defined for the image data points.

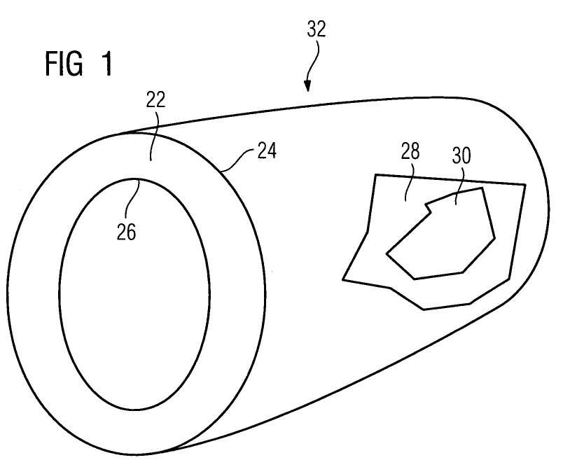

The image data record containing the necrotic parts of the myocardium is thus available on the one hand (two-dimensional representation shown as image 12 in FIG. 2) and, on the other, either the endocardium image data record (with image 10) or the myocardium image data record (see further down regarding FIG. 1). A registering step is then carried out. The method of registering is as such known in the prior art. Through recognizing common structures in the image data records being registered with each other it is determined how (which is to say according to which computing rule) the two image data records can be imaged one over the other. The relevant systems of coordinates do not, a priori, mutually tally by virtue of either their origin or their orientation because totally different image data records have been obtained so that the 3D image data has to be mutually assigned accurately in terms of position and dimensions.

As the result of registering it is possible to superimpose the partial images one on the other with the aid of the computing rule obtained. FIG. 2 illustrates superimposing of the partial image 10 (based on the endocardium image data record) on the partial image 12 (based on the second 3D image data record). What is obtained is the overall image 14, an image in which is shown on the one hand the endocardium 16 and, on the other, as a closed region, the necrotic myocardium 18.

Image 14 illustrates very clearly how very well the inventive method can support a doctor providing treatment: The position of the necrotic myocardium 18 is very precisely known in relation to the endocardium 16. The doctor knows in particular where a boundary 20 of the necrotic myocardium is situated. By virtue already of image 14, the doctor will be able to move up to boundary 20 with absolute precision during an electrophysiological procedure using, for example, an ablation catheter.

An alternative representation does not show the endocardium with the necrotic myocardium superimposed thereon; the myocardium image data record that is the result of segmenting the first 3D image data record is used instead. The symbolically represented healthy myocardium 22 with the outer myocardium 24 and inner myocardium 26 can be seen in FIG. 1, with the inner myocardium 26 defining the endocardium. Part 22 of the myocardium is shown in white, intended to symbolize that this is healthy myocardium. The region in which a myocardial infarction has occurred is emphasized. By virtue of the first 3D image data record's data being shown having the second 3D image data record superimposed thereon, both the myocardium 28 having a reduced blood supply and the necrotic myocardium 30 are shown. Let it be assumed that both are shown in different colors—indicated in FIG. 1 by different gray-scale values—, with the representation in different colors being made possible through the use of attributes. By virtue of the threshold-examining step performed during segmenting, it has also been made possible to differentiate between healthy myocardium (white) and myocardium 28 having a reduced blood supply.

FIG. 1 is thus a composite image 32 illustrating an alternative to image 14 according to FIG. 2. Thus either the necrotic myocardium is superimposed on the endocardium (image 14) or an image of the other parts of the myocardium is superimposed (image 32).

The image representation is in both cases by itself already useful for the doctor.

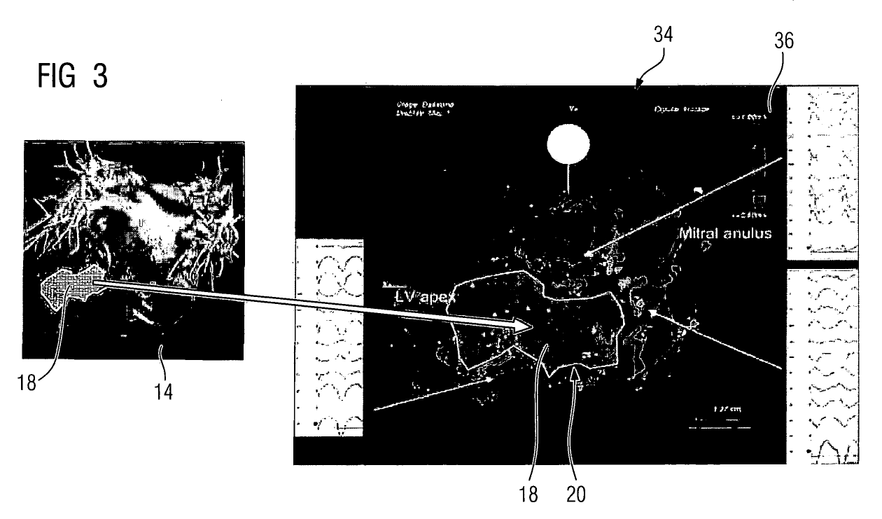

FIG. 3 illustrates a further step: The starting point therein is image 14. Image 14 preceded through the merging of two pre-operatively produced 3D image data records.

Let it be assumed that during an intervention using, for instance, an ablation catheter, the doctor providing treatment orientates himself/herself with the aid of, for example, an electroanatomical map, identified in FIG. 3 by the numeral 34. An electroanatomical map 34 is made by moving along different points in the endocardium using a probe (catheter) and in each case recording specific signals bearing a relationship with the heart's electrophysiological reactions. The scale 36 on the right in the image symbolizes different strengths of the signals, and the core region of the map 34 illustrates a two-dimensional representation of the respective strength.

It is again not yet possible a priori to correlate image 14 with image 34. It is, though, possible to repeat a registering step. Electroanatomical maps, too, contain image structures that can be correlated by means of automatic image recognition with image structures in image 14. That makes it possible to assign the systems of coordinates to each other, and what is again obtained is an imaging rule, or in other words a computing rule, for how the coordinates of one image system by means of which image 14 is produced can be translated into the coordinates of the other image system by means of which image 34 is produced.

It is alternatively also possible to take X-ray images during the operation, for example to carry out a 3D cardiac X-ray angiography (see DE 10 2005 016472.2, published subsequently). The superimposed image data from which image 14 was produced can then be registered with the X-ray images. As the catheter system with the aid of which an electroanatomical map 34 is produced is stationary relative to the X-ray system, said registering, which causes coordinates of the pre-operatively produced image data record to be assigned to the coordinates of the X-ray image data record produced during the intervention, can simultaneously allow the coordinates of the pre-operatively open image data record to be assigned to those of the electroanatomical system.

Regardless of how the second registering step is performed, it is possible to enter the region 18 of necrotic myocardium into the electroanatomical maps 34. That is illustrated in FIG. 3.

Although it may have been a long road to said superimposition of images, comprising as it does two registering steps and the production of a plurality of image data records, the methods requiring to be carried out are so reliable that the doctor providing treatment can find them useful and apply them in the course of his/her operation.

The boundaries 20 of the necrotic myocardium can by means of the representation according to FIG. 3 be directly correlated with contours in the electroanatomical map 34. As a catheter is moved in any event and electrophysiological systems already provide for automatic catheter movements, it is perfectly possible as a further step to provide for a catheter to be moved along the boundary line 20, specifically more or less automatically, with the doctor providing treatment then having only to in each case take care of guiding the system.

An automatic treatment system has thus been produced from the pure visualization.

At each of its stages (producing image 14 or image 32, producing image 34 having the region 18, automatically controlling the catheter) the invention constitutes a significant step for optimizing the treatment of myocardial infarctions.

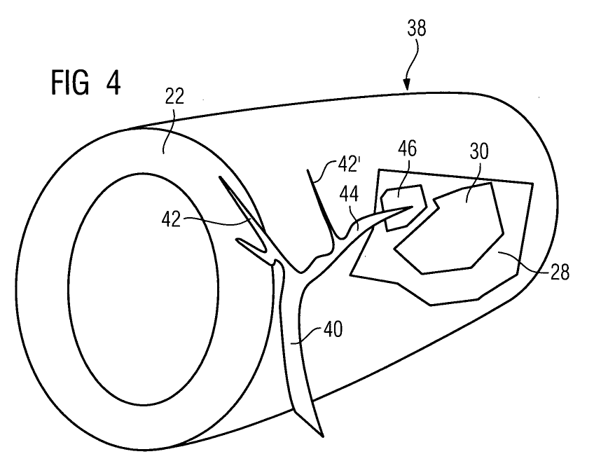

Myocardial infarctions can be treated also by selectively treating the coronary arteries. Here, too, it will already be advantageous for image 32, produced pre-operatively, to be available to the doctor providing treatment because, by virtue of his/her experience, the doctor will then be able to form a rough picture of where the various regions, to be seen in image 32, of myocardium (22, 28, 30) are situated in relation to a coronary artery of which he/she generally produces an image using a C-arm X-ray system. A coronary artery is imaged by inserting a catheter into the heart up to the start of the artery and selectively injecting a contrast medium into the artery. X-ray images are then taken. Virtually only the respective coronary artery and its ramifications can then be recognized in said angiograms. Here, too, the present invention can now go a step further: An image of a coronary artery can be superimposed on image 32 (FIG. 1). An image of said kind is shown in FIG. 4 and identified overall by the numeral 38.

What can be seen, as in image 32, is the healthy myocardium 22, the myocardium 28 having a reduced blood supply, and the necrotic myocardium 30. Additionally shown superimposed is a coronary artery 40 with ramifications 42 and 42′, with one branch 44 of the coronary artery 40 supplying blood to a section 46 of the myocardium 28 having a reduced blood supply. A representation according to image 38 is made possible through superimposing of a corresponding X-ray image (angiogram) of the coronary artery 40 on image 32. An additional registering step is for that purpose generally carried out, with registering being made possible through the myocardium 28 that has a reduced blood supply (in particular the region 46) being partially shown in the angiogram, or also through parts of the healthy myocardium 22 being weakly visible therein, with the contours then enabling the two images' coordinates to be assigned accurately in terms of position and dimensions. Registering can be omitted if image 32 was for its part produced using the same C-arm X-ray system, with the patient then not being allowed to have moved while image 32 was being produced and up until imaging of the coronary artery 40.

Image 38 is a further stage in supporting the doctor during interventions on the coronary arteries. He/she will be able to identify the branch 44 as being close to the myocardium 46 having a reduced blood supply, and selectively begin a treatment in the branch 44 to lessen the reduction in blood supply to the region 46.

The image of the coronary artery 40 can derive from a 2D image (2D angiogram) or be a representation of a 3D image data record.

1-11. (canceled)

12. A method for imaging a myocardium of a patient having an infarction, comprising:
generating a first image data record immediately after administrating a contrast medium to the patient, the first image data record comprising image information of an endocardium, a healthy part of the myocardium, and a part of the myocardium having a reduced blood supply;
segmenting the first image data record to a segmented first image data record comprising an image selected from the group consisting of: the endocardium, the healthy part of the myocardium, and the part of the myocardium having the reduced blood supply;
generating a second image data record when a pre-specified period of time has elapsed after the administration of the contrast medium, the second image data record comprising an image of a necrotic part of the myocardium;
registering the second image data record with the segmented first image data record;
superimposing the second image data record with the segmented first image data record based on the registration to produce an overall image data record;
generating an overall image representation comprising the image of the necrotic part of the myocardium and the image in the segmented first image data record; and
using the overall image representation in a humanly perceptible manner.

13. The method as claimed in claim 12, wherein the segmented first image data record is an endocardium image data record comprising an image of the endocardium or a myocardium image data record comprising the image of the health part of the myocardium and the part of the myocardium having the reduced blood supply.

14. The method as claimed in claim 12,
wherein the healthy part of the myocardium, the part of the myocardium having the reduced blood supply, and the necrotic part of the myocardium are assigned to three different attributes and are shown differently in the overall image representation, and
wherein the parts having different attributes are shown differently in the overall image representation with different colors.

15. The method as claimed in claim 12, wherein the first and the second image data records are three-dimensional and the overall image representation is two-dimensional.

16. The method as claimed in claim 12, wherein the first and the second image data records are recorded by a C-arm X-ray system.

17. The method as claimed in claim 12, wherein the first and the second image data records are generated at a same pre-specified cardiac or respiration phase.

18. A method for supporting a therapeutic intervention on a heart of a patient, comprising:
generating a first image data record immediately after administrating a contrast medium to the patient before the therapeutic intervention, the first image data record comprising image information of an endocardium, a healthy part of a myocardium, and a part of the myocardium having a reduced blood supply;
segmenting the first image data record to a segmented first image data record comprising an image selected from the group consisting of: the endocardium, the healthy part of the myocardium, and the part of the myocardium having the reduced blood supply;
generating a second image data record when a pre-specified period of time has elapsed after the administration of the contrast medium before the therapeutic intervention, the second image data record comprising an image of a necrotic part of the myocardium;
registering the second image data record with the segmented first image data record;
superimposing the second image data record with the segmented first image data record based on the registration to produce a pre-operative overall image data record;
generating an operation image during the therapeutic intervention;
superimposing the operation image with the pre-operative overall image data record;
generating an image representation with an image in the pre-operatively overall image data record superimposed on the operation image; and
using the image representation during the therapeutic intervention.

19. The method as claimed in claim 18, wherein the image in the pre-operatively overall image data record is the image selected from the group consisting of: the healthy part of the myocardium, the part of the myocardium having the reduced blood supply, the necrotic part of the myocardium, and a combination thereof.

20. The method as claimed in claim 18,
wherein a boundary between the necrotic part of the myocardium and other areas of tissue is determined from the image representation and a therapy instrument is automatically moved to a point on the boundary, and
wherein the therapy instrument is an ablation catheter and is automatically moved to the point along the boundary.

21. The method as claimed in claim 18, wherein the operation image is a two-dimensional image generated by visualizing a three-dimensional electroanatomical mapping data in the two-dimensional image.

22. The method as claimed in claim 18, wherein the operation image is a three-dimensional operation image data record using a three-dimensional heart rotation angiography.

23. The method as claimed in claim 18,
wherein the operation image is a two-dimensional angiogram or a three-dimensional rotation angiogram using a C-arm X-ray system and is generated after injecting a contrast medium into a coronary artery, and
wherein the coronary artery in the operation image is shown in the image representation simultaneously with the image in the pre-operatively overall image data record.

24. The method as claimed in claim 18, wherein the first image data record, the second image data record, and the operation image are generated at a same pre-specified cardiac or respiration phase.

25. The method as claimed in claim 18,
wherein the first image data record, the second image data record, and the operation image are recoded by a same image system with the patient having a same position in the image system during recording, and
wherein the image system is a C-arm X-ray image system.

26. The method as claimed in claim 18, wherein the operation image is registered with the pre-operative overall image data record before superimposing the operation image with the pre-operative overall image data record if the first and the second image data records and the operation image are recoded by different image systems or the patient has moved in the image system during recording.

27. A medical system for imaging a myocardium of a patient having an infarction, comprising:
an image system that records:
a first image data record immediately after administrating a contrast medium to the patient, the first image data record comprising image information of an endocardium and a healthy part of the myocardium and a part of the myocardium having a reduced blood supply,
a second image data record when a pre-specified period of time has elapsed after the administration of the contrast medium, the second image data record comprising an image of a necrotic part of the myocardium; and
an image processing device that:
segments the first image data record to a segmented first image data record comprising an image selected from the group consisting of: the endocardium, the healthy part of the myocardium, and the part of the myocardium having the reduced blood supply,
registers the second image data record with the segmented first image data record,
generates an overall image data record by superimposing the second image data record with the segmented first image data record based on the registration, and
generates an overall image representation comprising the image of the necrotic part of the myocardium and the image in the segmented first image data record.

28. The medical system as claimed in the claim 27,
wherein the first and the second image data records are recorded before a therapeutic intervention and an operation image is recorded during the therapeutic intervention, and
wherein the operation image is superimposed with the pre-operatively overall image data record.

29. The medical system as claimed in the claim 28, wherein the first image data record, the second image data record, and the operation image are recorded at a same pre-specified cardiac or respiration phase.

30. The medical system as claimed in the claim 28,
wherein the first image data record, the second image data record, and the operation image are recoded by a same image system with the patient having a same position in the image system during recording, and
wherein the image system is a C-arm X-ray image system.

31. medical system as claimed in the claim 28, wherein the operation image is registered with the pre-operative overall image data record before superimposing the operation image with the pre-operative overall image data record if the first and the second image data records and the operation image are recoded by different image systems or the patient has moved in the image system during recording.
